(19) United States
(12) Patent Application Publication
(10) Pub. No.: US 2025/0129218 A1
(43) Pub. Date: Apr. 24, 2025

(54) HYDROGEL KIT CAPABLE OF BEING QUICKLY DISSOLVED AS REQUIRED AND USE METHOD THEREOF

(71) Applicant: SHANGHAI RUINING BIOTECHNOLOGY CO., LTD., Shanghai (SH)

(72) Inventors: Liang CHEN, Shanghai (CN); Zhen PAN, Shanghai (CN)

(21) Appl. No.: 19/007,584

(22) Filed: Jan. 2, 2025

Related U.S. Application Data
(63) Continuation of application No. PCT/CN2024/077043, filed on Feb. 8, 2024.

(30) Foreign Application Priority Data
Aug. 22, 2023 (CN) 202311064236.7

(51) Int. Cl. C08J 3/075

(52) U.S. Cl. CPC C08J 3/075; C08J 2305/02; C08J 2371/02; C08J 2477/04; C08J 2479/02

(57) ABSTRACT

The present disclosure discloses a hydrogel kit capable of being quickly dissolved on demand, which includes a gel system and a dissolving solution, and a use volume ratio of the gel system to the dissolving solution is 1:(2-10). According to the present disclosure, under the combined action of an aldehyde group-terminated star-shaped multi-arm polyethylene glycol gel system and a water-soluble amino compound dissolving solution, rapid degradation of a gel can be achieved, gel degradation is achieved within half an hour, and the problem that inflammations are caused to the body after functional requirements of the gel are met is avoided. Moreover, the pH value of the water-soluble amino compound dissolving solution is controlled to be 3-7.5, such that low-temperature rapid degradation of the gel system can be achieved, and the degradation time is controlled to be less than 30 min.

### CROSS-REFERENCE TO RELATED APPLICATIONS

The present application is a continuation of International Application No. PCT/CN2024/077043, with an international filing date of Feb. 8, 2024, which is based upon and claims priority to Chinese Patent Application No. 2023110642367, filed on Aug. 22, 2023, the entire contents of all of which are incorporated herein by reference.

### TECHNICAL FIELD

The present disclosure relates to a hydrogel kit capable of being quickly dissolved as required and a use method thereof, relates to A61L, and specifically relates to the field of pharmaceutical preparations.

### BACKGROUND

Hydrogels are a kind of polymer materials with high water contents and good biocompatibility, and have been gradually used in the field of medicine in recent years. Due to the good biocompatibility, the hydrogels have been widely used in the fields of tissue spacing, tissue repair, drug sustained release, wound dressings, medical cosmetology, scar repair, etc. Due to their gel characteristics, the hydrogels have low degradation speeds in the body. When the gels cannot be quickly removed after fulfilling their medical functions, they might stay in the body for a long time, leading to inflammations caused by foreign matter in the body. In addition, the hydrogels need to be quickly removed after completing functional tasks in tissue spacing, wound dressings and scar repair, otherwise further repair and growth of body tissues will be affected. The hydrogels may also have deviations of implantation positions when used in vivo or in vitro, in which case the hydrogels will need to be removed. However, due to the causes that most of the hydrogels are implanted by a minimally invasive in-situ injection method or the gels have strong adhesion to tissues at implantation sites, the hydrogels may exhibit relatively large resistance to removal. Therefore, how to quickly and harmlessly remove the hydrogels is a technical problem to be solved urgently in clinical applications.

Chinese Invention Patent CN115737535A discloses a controllable and degradable composite nanogel as well as a preparation method therefor and use thereof. An aminophenylboronic acid modified hyaluronic acid-crosslinked polyvinyl alcohol/hydrogel-liposome regulates internal configuration changes through a dynamic aminophenylborate covalent bond and a hyaluronic acid skeleton under the action of hydrolysis to complete automatic degradation after the release of a nanodrug, thereby achieving precise clinical treatment. However, the composite nanogel has a relatively long degradation time and has the risk of foreign matter inflammations. Chinese Invention Patent CN202011119562.X discloses a degradable medical hydrogel. By using aldehyde group-terminated star-shaped multi-arm polyethylene glycol and optimizing the arm number and molecular weight range of the star-shaped multi-arm polyethylene glycol, a hydrogel capable of being degraded within a short time is obtained. However, the degradation still takes several days to one year, and the degradation speed is low.

### SUMMARY

In order to improve degradation speeds of hydrogels and achieve rapid and harmless dissolution, a first aspect of the present disclosure provides a hydrogel kit capable of being quickly dissolved as required, which includes a gel system and a dissolving solution, and a use volume ratio of the gel system to the dissolving solution is 1:(2-10).

As one preferred embodiment, the gel system includes an aldehyde-derivative aqueous solution and a polyamino aqueous solution, and a volume ratio of the aldehyde-derivative aqueous solution to the polyamino aqueous solution is 1:(0-10).

As one preferred embodiment, the volume ratio of the aldehyde-derivative aqueous solution to the polyamino aqueous solution is 1:(0.5-2).

As one preferred embodiment, the aldehyde-derivative aqueous solution is selected from a combination of one or more of a polyethylene glycol aldehyde-derivative aqueous solution, an oxidized sodium carboxymethyl cellulose aqueous solution, an oxidized sodium alginate aqueous solution, and an oxidized dextran sulphate sodium aqueous solution.

As one preferred embodiment, the polyethylene glycol aldehyde-derivative aqueous solution is aldehyde group-terminated star-shaped multi-arm polyethylene glycol, and an arm number of the aldehyde group-terminated star-shaped multi-arm polyethylene glycol is 4-8.

As one preferred embodiment, a weight-average molecular weight of the aldehyde group-terminated star-shaped multi-arm polyethylene glycol is 2,000-5,000 Da.

As one preferred embodiment, the aldehyde group is selected from a combination of one or two of an aromatic aldehyde group and an alkyl aldehyde group. Further preferably, the aldehyde group is a phenyl aldehyde group.

As one preferred embodiment, bonding of the aldehyde group to the star-shaped multi-arm polyethylene glycol is selected from a combination of one or several of an ether bond, an amide bond, an urethane bond, an imine bond, and a urea bond.

As one preferred embodiment, a mass fraction of the polyethylene glycol aldehyde-derivative aqueous solution is 5-50%; and a mass fraction of the polyamino aqueous solution is 0.5-30%.

As one preferred embodiment, the mass fraction of the phenyl aldehyde derivatized polyethylene glycol aqueous solution is 15-30%; and the mass fraction of the polyamino aqueous solution is 1-6%.

As one preferred embodiment, the mass fraction of the phenyl aldehyde derivatized polyethylene glycol aqueous solution is 20-25%; and the mass fraction of the polyamino aqueous solution is 1-5%.

As one preferred embodiment, the mass fraction of the phenyl aldehyde derivatized polyethylene glycol aqueous solution is 20%; and the mass fraction of the polyamino aqueous solution is 1-5%.

As one preferred embodiment, the polyamino aqueous solution is selected from a combination of one or two of a polyethylenimine aqueous solution and a polylysine aqueous solution.

As one preferred embodiment, a mass fraction of the polyethylenimine aqueous solution is 0.5-10%, and a mass fraction of the polylysine aqueous solution is 1-30%.

As one preferred embodiment, the mass fraction of the polyethylenimine aqueous solution is 1-6%, and the mass fraction of the polylysine aqueous solution is 1.5-4.5%.

As one preferred embodiment, the mass fraction of the polyethylenimine aqueous solution is 1-5%, and the mass fraction of the polylysine aqueous solution is 2-3%.

As one preferred embodiment, when the polyamino aqueous solution only includes the polyethylenimine aqueous solution, the mass fraction of the polyethylenimine aqueous solution is 5%. In the gel system, a volume ratio of the polyethylene glycol aldehyde-derivative aqueous solution to the polyethylenimine aqueous solution is 1:1.

As one preferred embodiment, when the polyamino aqueous solution includes a combination of the polyethylenimine aqueous solution and the polylysine aqueous solution, the mass fraction of the polyethylenimine aqueous solution is 1.2%, and the mass fraction of the polylysine aqueous solution is 2.6%. In the gel system, a volume ratio of the polyethylene glycol aldehyde-derivative aqueous solution, the polyethylenimine aqueous solution to the polylysine aqueous solution is 1:1:1.

The applicant has found in an experimental process that under the combined action of the gel system and the water-soluble amino compound dissolving solution, rapid degradation of a gel can be achieved in a certain acidic or alkaline environment, gel degradation is achieved within half an hour, and the problem that inflammations are caused to the body after functional requirements of the gel are met can be avoided. Possible reasons are speculated as follows: an amide bond-crosslinked gel network is formed after an aldehyde group-terminated multi-arm polyethylene glycol derivative undergoes a Schiff base reaction with a polyamino compound, where a crosslinking point in the gel network is a dynamic amide bond, and a single-molecule amino group in the introduced water-soluble amino compound dissolving solution can break the dynamic amide bond in a suitable acidic or alkaline environment, such that a crosslinked network structure is broken, dissolved in the solution and then discharged out of the body.

As one preferred embodiment, the dissolving solution is selected from a combination of one or several of a hydroxylamine hydrochloride aqueous solution, an amino acid aqueous solution, and a short peptide aqueous solution.

As one preferred embodiment, the dissolving solution is selected from one of a hydroxylamine hydrochloride aqueous solution, a glycine aqueous solution, and a lysine aqueous solution.

As one preferred embodiment, the dissolving solution is a hydroxylamine hydrochloride aqueous solution; preferably, the dissolving solution is a glycine aqueous solution; and preferably, the dissolving solution is a lysine aqueous solution.

As one preferred embodiment, a concentration of the dissolving solution is 0.1-5 wt %, and preferably, the concentration of the dissolving solution is one of 0.1 wt %, 0.5 wt %, 2 wt %, and 5 wt %.

As one preferred embodiment, a pH value of the dissolving solution before dissolution is 1-7.5, and a pH value of the dissolving solution after dissolution is 3-8.

Preferably, the pH value of the dissolving solution before dissolution is 3-7.5, and the pH value of the dissolving solution after dissolution is 3-8.

The applicant has further found that the multi-arm polyethylene glycol derivative has a negatively charged group that has different bonding strengths with an ionic bond of the polyamino compound under different pH conditions, and rapid degradation of the gel system can be achieved through the water-soluble amino compound at the pH value of 3-7.5.

As one preferred embodiment, a dissolution time of the gel system in the hydrogel kit is 2-210 min.

As one preferred embodiment, the dissolution time of the gel system in the hydrogel kit is 2-30 min.

As one preferred embodiment, a dissolution temperature of the gel system in the hydrogel kit is 20-37° C.

As one preferred embodiment, the hydrogel kit capable of being quickly dissolved as required can be used in preparation of one of tissue fillers, tissue adhesion barriers, tissue engineering scaffolds, sealing agents, embolic agents, drug carrier materials, skin dressings, radiotherapy spacers, postoperative tissue sealing, and anti-leakage agents, etc.

A second aspect of the present disclosure provides a use method of the hydrogel kit capable of being quickly dissolved as required, which includes the following steps:
(1) mixing the aldehyde derivatized polyethylene glycol aqueous solution with the polyamino aqueous solution at a corresponding volume ratio to form a gel system;
(2) mixing the gel system with the dissolving solution at a volume ratio, and adjusting the pH value of the system; and
(3) testing a degradation time.

Compared with the prior art, the present disclosure has the following beneficial effects.
(1) According to the hydrogel kit capable of being quickly dissolved on demand of the present disclosure, under the combined action of the aldehyde group-terminated star-shaped multi-arm polyethylene glycol gel system and the water-soluble amino compound dissolving solution, rapid degradation of a gel can be achieved, gel degradation is achieved within half an hour, and the problem that inflammations are caused to the body after functional requirements of the gel are met is solved.
(2) According to the hydrogel kit capable of being quickly dissolved on demand of the present disclosure, the pH value of the water-soluble amino compound dissolving solution is controlled to be 1-7.5, such that low-temperature rapid degradation of the gel system can be achieved, and the degradation time is controlled to be less than 30 min.
(3) According to the hydrogel kit capable of being quickly dissolved on demand of the present disclosure, rapid degradation can be achieved within half an hour at a mild temperature in a mild acidic or alkaline environment, the application range of the gel is greatly widened, and side effects caused by long-term stimulation of foreign matter to patients are reduced.

### BRIEF DESCRIPTION OF THE DRAWINGS

FIG. 1 is a picture of a gel system in Example 17 before dissolution;

FIG. 2 is a picture of the gel system in Example 17 in dissolution; and

FIG. 3 is a picture of the gel system in Example 17 after dissolution.

### DESCRIPTION OF THE EMBODIMENTS

### Example

A hydrogel kit capable of being quickly dissolved on demand and a use method thereof are provided. The hydrogel kit includes a gel system and a dissolving solution, and a use volume ratio of the gel system to the dissolving solution is 1:5.

The gel system I includes 20 wt % of a phenyl aldehyde derivatized polyethylene glycol aqueous solution and 5 wt % of a polyethylenimine aqueous solution at a volume ratio of 1:1.

The gel system II includes 20 wt % of a phenyl aldehyde derivatized polyethylene glycol aqueous solution, 1.2 wt % of a polyethylenimine aqueous solution and 2.6 wt % of a polylysine aqueous solution at a volume ratio of 1:1:1.

The gel system III includes 20 wt % of an aldehyde group oxidized dextran aqueous solution and 10 wt % of a polyethylenimine aqueous solution at a volume ratio of 1:1.

The gel system IV includes 20 wt % of an aldehyde group oxidized dextran aqueous solution, 1.2 wt % of polyethylenimine and 2.6 wt % of a polylysine aqueous solution at a volume ratio of 1:1:1.

The phenyl aldehyde derivatized polyethylene glycol aqueous solution is a phenyl aldehyde group-terminated star-shaped multi-arm polyethylene glycol solution with an arm number of 4 and a weight-average molecular weight of 2,000-5,000 Da, which was purchased from Beijing Jenkem Technology Co., Ltd. The aldehyde group oxidized dextran was purchased from Shanghai Reunion Biotechnology Co., Ltd. The polyethylenimine was purchased from Shanghai Macklin Biochemical Co., Ltd. The polylysine was purchased from Shanghai Macklin Biochemical Co., Ltd.

Raw materials for preparation, use process conditions, and the degradation time are shown in Table 1.

TABLE 1

| | | | | | | pH of the | |
|---|---|---|---|---|---|---|---|
| | | | | | | dissolving | |
| | | | pH of the | Gel | | solution | |
| | | Dissolving | dissolving | weight | Degradation | after | Degradation |
| | Gel system | solution | solution | (g) | temperature | degradation | time |
| Example 1 | Gel system | PBS | 3 | 0.5002 | 37° C. | 4.41 | Degraded |
| | I | | | | | | within 2.5 h |
| Example 2 | Gel system | PBS | 4 | 0.5039 | 37° C. | 5.49 | Degraded |
| | I | | | | | | within 3 h |
| Example 3 | Gel system | PBS | 6 | 0.5334 | 37° C. | 6.43 | Degraded |
| | I | | | | | | within 17 h |
| Example 4 | Gel system | PBS | 7.4 | 0.5221 | 37° C. | 7.78 | Degraded |
| | I | | | | | | within 120 h |
| Example 5 | Gel system | PBS | 9 | 0.5314 | 37° C. | 9.64 | Degraded |
| | I | | | | | | within 123 h |
| Example 6 | Gel system | 0.5 wt % | 7.43 | 0.5118 | 37° C. | 7.75 | Degraded |
| | I | hydroxylamine | | | | | within 3.5 h |
| | | hydrochloride | | | | | |
| Example 7 | Gel system | 2 wt % | 7.42 | 0.521 | 37° C. | 7.71 | Degraded |
| | I | hydroxylamine | | | | | within 3 h |
| | | hydrochloride | | | | | |
| Example 8 | Gel system | 0.5 wt % | 7.45 | 0.5113 | 37° C. | 7.58 | Degraded |
| | I | glycine | | | | | within 70 h |
| Example 9 | Gel system | 2 wt % | 7.44 | 0.5002 | 37° C. | 7.5 | Degraded |
| | I | glycine | | | | | within 70 h |
| Example 10 | Gel system | PBS | 3 | 0.5112 | 37° C. | 5.09 | Degraded |
| | II | | | | | | within 3 h |
| Example 11 | Gel system | PBS | 7.4 | 0.5 | 37° C. | / | Not degraded |
| | II | | | | | | within 90 days |
| Example 12 | Gel system | PBS | 9 | 0.5372 | 37° C. | / | Not degraded |
| | II | | | | | | within 96 h |
| Example 13 | Gel system | 0.5 wt % | 7.43 | 0.5083 | 37° C. | 7.61 | Degraded |
| | II | hydroxylamine | | | | | within 4 h |
| | | hydrochloride | | | | | |
| Example 14 | Gel system | 2 wt % | 7.42 | 0.5009 | 37° C. | 7.45 | Degraded |
| | II | hydroxylamine | | | | | within 3.5 h |
| | | hydrochloride | | | | | |
| Example 15 | Gel system | 0.5 wt % | 7.45 | 0.5298 | 37° C. | / | Not degraded |
| | II | glycine | | | | | within 96 h |
| Example 16 | Gel system | 2% | 7.44 | 0.511 | 37° C. | / | Not degraded |
| | II | glycine | | | | | within 96 h |
| Example 17 | Gel system | 0.5% | 3.49 | 0.641 | 37° C. | 3.94 | Degraded |
| | II | hydroxylamine | | | | | within 4 min |
| | | hydrochloride | | | | | |
| Example 18 | Gel system | 2 wt % | 3.42 | 0.841 | 37° C. | 3.5 | Degraded |
| | II | hydroxylamine | | | | | within 3 min |
| | | hydrochloride | | | | | |
| Example 19 | Gel system | 0.5 wt % | 4.07 | 0.564 | 37° C. | 4.17 | Degraded |
| | II | glycine | | | | | within 15 min |
| Example 20 | Gel system | 2 wt % | 4.02 | 0.714 | 37° C. | 4.08 | Degraded |
| | II | glycine | | | | | within 18 min |
| Example 21 | Gel system | 0.5 wt % | 3.49 | 0.5 | 20° C. | 4.01 | Degraded |
| | II | hydroxylamine | | | | | within 3.5 min |
| | | hydrochloride | | | | | |
| Example 22 | Gel system | 2 wt % | 3.42 | 0.5 | 20° C. | 3.3 | Degraded |
| | II | hydroxylamine | | | | | within 2.5 min |
| | | hydrochloride | | | | | |
| Example 23 | Gel system | 2 wt % | 5.03 | 0.4951 | 37° C. | 4.89 | Degraded |
| | II | hydroxylamine | | | | | within 12 min |
| | | hydrochloride | | | | | |
| Example 24 | Gel system | 2 wt % | 6.02 | 0.4873 | 37° C. | 5.97 | Degraded |
| | II | hydroxylamine | | | | | within 18 min |
| | | hydrochloride | | | | | |
| Example 25 | Gel system | 0.1 wt % | 4.04 | 0.5002 | 37° C. | 3.94 | Degraded |
| | II | hydroxylamine | | | | | within 25 min |
| | | hydrochloride | | | | | |
| Example 26 | Gel system | 5 wt % | 3.99 | 0.4799 | 37° C. | 4.32 | Degraded |
| | II | hydroxylamine | | | | | within 8 min |
| | | hydrochloride | | | | | |
| Example 27 | Gel system | 2 wt % | 3.02 | 0.5009 | 37° C. | 3.2 | Degraded |
| | II | glycine | | | | | within 15 min |
| Example 28 | Gel system | 5 wt % | 3.01 | 0.4556 | 37° C. | 3.14 | Degraded |
| | II | glycine | | | | | within 12 min |
| Example 29 | Gel system | 2 wt % | 3 | 0.5147 | 37° C. | 3.2 | Degraded |
| | II | lysine | | | | | within 23 min |
| Example 30 | Gel system | 5 wt % | 3 | 0.5229 | 37° C. | 3.22 | Degraded |
| | II | lysine | | | | | within 28 min |
| Example 31 | Gel system | 2 wt % | 5.03 | 0.5446 | 37° C. | 5.11 | Degraded |
| | II | lysine | | | | | within 28 min |
| Example 32 | Gel system | 5 wt % | 5.01 | 0.5091 | 37° C. | 5.16 | Degraded |
| | II | lysine | | | | | within 28 min |
| Example 33 | Gel system | 2 wt % | 7.03 | 0.5274 | 37° C. | / | Not degraded |
| | II | lysine | | | | | within 18 h |
| Example 34 | Gel system | PBS | 7.4 | 0.5078 | 37° C. | / | Not degraded |
| | III | | | | | | within 9 h |
| Example 35 | Gel system | 0.5 wt % | 3.49 | 0.512 | 37° C. | 3.52 | Degraded |
| | III | hydroxylamine | | | | | within 2 min |
| | | hydrochloride | | | | | |
| Example 36 | Gel system | 2 wt % | 3.42 | 0.461 | 37° C. | 3.45 | Degraded |
| | III | hydroxylamine | | | | | within 2 min |
| | | hydrochloride | | | | | |
| Example 37 | Gel system | PBS | 7.4 | 0.5078 | 37° C. | / | Not degraded |
| | IV | | | | | | within 9 h |
| Example 38 | Gel system | 0.5 wt % | 3.49 | 0.4596 | 37° C. | 3.63 | Degraded |
| | IV | hydroxylamine | | | | | within 3 min |
| | | hydrochloride | | | | | |
| Example 39 | Gel system | 2 wt % | 3.42 | 0.4603 | 37° C. | 3.46 | Degraded |
| | IV | hydroxylamine | | | | | within 3 min |
| | | hydrochloride | | | | | |

The PBS is a buffer solution containing sodium dihydrogen phosphate and disodium hydrogen phosphate.

A gel of the gel system II can maintain a gel form after degradation in the PBS buffer solution with a pH value of 7.4 for 90 days, which has a long degradation time. After an acidic solution containing an amino group is added, the gel can be dissolved within 2-3 min. The application range of the gel is greatly widened, and side effects caused by long-term stimulation of foreign matter to patients are reduced.

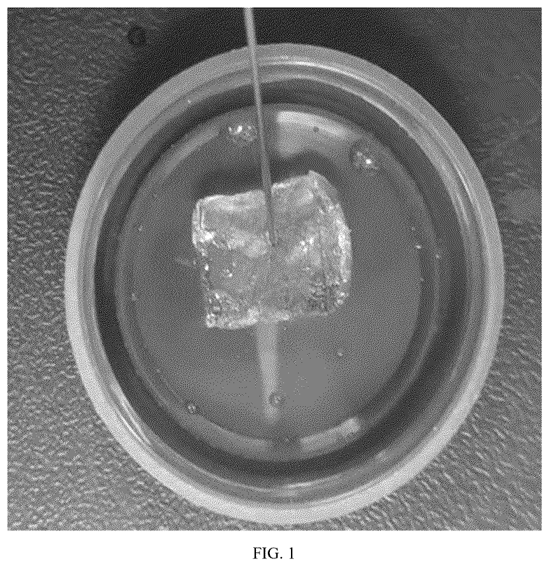

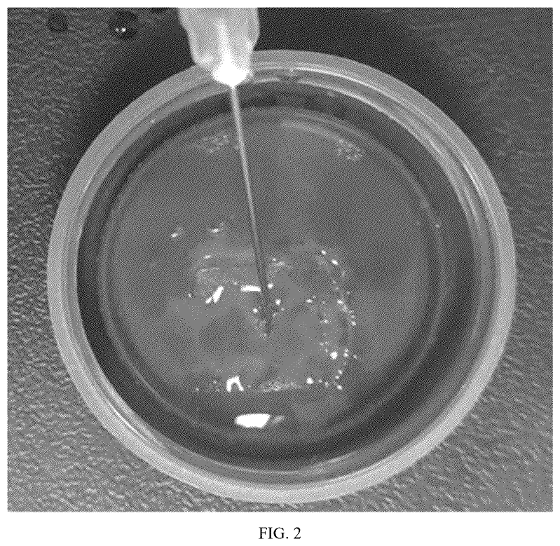

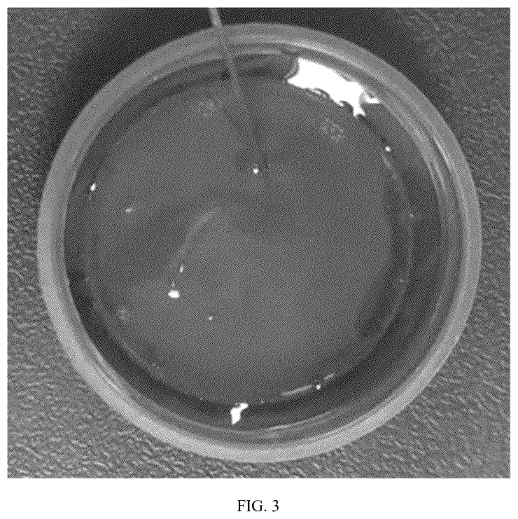

A dissolution process of the gel system in Example 17 is shown in FIGS. 1-3.

What is claimed is:

1. A hydrogel kit capable of being quickly dissolved on demand, wherein the hydrogel kit comprises a gel system and a dissolving solution, and a use volume ratio of the gel system to the dissolving solution is 1:(2-10).

2. The hydrogel kit of claim 1, wherein the gel system comprises an aldehyde-derivative aqueous solution and a polyamino aqueous solution, and a volume ratio of the aldehyde-derivative aqueous solution to the polyamino aqueous solution is 1:(0-10).

3. The hydrogel kit of claim 2, wherein the aldehyde-derivative aqueous solution is selected from a combination of one or more of a aldehyde derivatized polyethylene glycol aqueous solution, an aldehyde group oxidized sodium carboxymethyl cellulose aqueous solution, an aldehyde group oxidized sodium alginate aqueous solution, and an aldehyde group oxidized dextran aqueous solution, the aldehyde derivatized polyethylene glycol aqueous solution is aldehyde group-terminated star-shaped multi-arm polyethylene glycol, and an arm number of the aldehyde group-terminated star-shaped multi-arm polyethylene glycol is 4-8.

4. The hydrogel kit of claim 3, wherein a mass fraction of the aldehyde derivatized polyethylene glycol aqueous solution is 5-50%; and a mass fraction of the polyamino aqueous solution is 0.5-30%.

5. The hydrogel kit of claim 2, wherein the polyamino aqueous solution is selected from a combination of one or two of a polyethylenimine aqueous solution and a polylysine aqueous solution.

6. The hydrogel kit of claim 5, wherein a mass fraction of the polyethylenimine aqueous solution is 0.5-10%, and a mass fraction of the polylysine aqueous solution is 1-30%.

7. The hydrogel kit of claim 5, wherein the dissolving solution is selected from a combination of one or several of a hydroxylamine hydrochloride aqueous solution, an amino acid aqueous solution, and a short peptide aqueous solution.

8. The hydrogel kit of claim 5, wherein a pH value of the dissolving solution before dissolution is 1-7.5, and a pH value of the dissolving solution after dissolution is 3-8.

9. The hydrogel kit of claim 5, wherein a dissolution time of the gel system in the hydrogel kit is 2-210 min.

10. A use method of the hydrogel kit of claim 4, comprising the following steps:
(1) mixing the polyethylene glycol aldehyde group derivative aqueous solution with the polyamino aqueous solution at a corresponding volume ratio to form a gel system;
(2) mixing the gel system with the dissolving solution at a volume ratio, and adjusting the pH value of the system; and
(3) testing a degradation time.
